(19) United States
(12) Patent Application Publication
(10) Pub. No.: US 2009/0319019 A1
(43) Pub. Date: Dec. 24, 2009

(54) Expandable Tip Delivery System For Endoluminal Prosthesis

(75) Inventor: Fred T. Parker, Unionville, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/487,501

(22) Filed: Jun. 18, 2009

Related U.S. Application Data
(60) Provisional application No. 61/074,788, filed on Jun. 23, 2008.

(51) Int. Cl. A61F 2/06

(52) U.S. Cl. 623/1.11

(57) ABSTRACT

An improved delivery system for an implantable medical device includes a retention sheath for an implantable medical device. The retention sheath includes a central lumen extending from a proximal end to a distal end of the retention sheath, and a tapered portion disposed at a distal end of the retention sheath. The tapered portion of the retention sheath includes a first layer made of a low-friction material. The first layer may be movable from a compressed, folded configuration in an initial position, to a substantially uncompressed and unfolded configuration in a deployment position. The retention sheath also includes a second layer made of an expandable material. The second layer is disposed radially outward of and in contact with the first layer, and the second layer is configured to expand in a substantially radially outward direction when the first layer moves from the initial position to the deployment position.

### RELATED APPLICATION

The present application claims the benefit of U.S. Provisional Application No. 61/074,788, filed on Jun. 23, 2008, the entirety of which is hereby incorporated by reference.

### BACKGROUND

1. Field of the Invention

The present invention relates generally to medical devices and more particularly to delivery systems for implantable medical devices, such as self-expanding stents.

2. Technical Background

Stents have become a common alternative for treating vascular conditions because stenting procedures are considerably less invasive than other alternatives. As an example, stenoses in the coronary arteries have traditionally been treated with bypass surgery. In general, bypass surgery involves splitting the chest bone to open the chest cavity and grafting a replacement vessel onto the heart to bypass the stenosed artery. However, coronary bypass surgery is a very invasive procedure that presents increased risk and requires a long recovery time for the patient. By contrast, stenting procedures are performed transluminally and do not require open surgery. Thus, recovery time is reduced and the risks of surgery are minimized.

Many different types of stents and stenting procedures are possible. In general, however, stents are typically designed as tubular support structures that may be inserted percutaneously and transluminally through a body passageway. Typically, stents are adapted to be compressed and expanded between a smaller and larger diameter. However, other types of stents are designed to have a fixed diameter and are not generally compressible. Although stents may be made from many types of materials, including non-metallic materials and natural tissues, common examples of metallic materials that may be used to make stents include stainless steel and nitinol. Other materials may also be used, such as cobalt-chrome alloys, amorphous metals, tantalum, platinum, gold, titanium, polymers and/or compatible tissues. Typically, stents are implanted within an artery or other passageway by positioning the stent within the lumen to be treated and then expanding the stent from a compressed diameter to an expanded diameter. The ability of the stent to expand from a compressed diameter makes it possible to thread the stent through narrow, tortuous passageways to the area to be treated while the stent is in a relatively small, compressed diameter. Once the stent has been positioned and expanded at the area to be treated, the tubular support structure of the stent contacts and radially supports the inner wall of the passageway. The implanted stent may be used to mechanically prevent the passageway from closing in order to keep the passageway open to facilitate fluid flow through the passageway.

Self-expanding stents are one common type of stent used in medical procedures. Self-expanding stents are increasingly being used by physicians because of their adaptability to a variety of different conditions and procedures. Self-expanding stents are usually made of shape memory materials or other elastic materials that act like a spring. Typical metals used in this type of stent include Nitinol and stainless steel. However, other materials may also be used.

To facilitate stent implantation, delivery catheters are widely used to deliver a stent or a stent graft to a deployment site in a patient's vasculature. Normally, stents are installed on the end of the delivery catheter inside a retention sheath in a low profile, compressed state. Delivery catheters used for self-expanding stents commonly include an inner catheter (inner core) that carries the stent. The inner catheter typically includes a distal tip that is atraumatic and that may be used to assist in dilating the vessel as the delivery system is advanced along a guide-wire that has been inserted into the patient's vasculature to the portion of the vessel to be treated. The distal tip commonly tapers radially in the distal direction from an outer diameter that substantially corresponds to the outer diameter of the distal end of the retention sheath, to a smaller outer diameter that substantially corresponds to the outer diameter of the guide-wire plus an appropriate wall thickness at the distal end of the distal tip. The distal tip may be bonded to the distal end of the inner catheter using an adhesive or the like.

Once the delivery catheter and stent are positioned adjacent the portion to be treated, the stent is released by pulling, or withdrawing, the sheath rearward. Normally, a stop or other feature is provided on the catheter to prevent the stent from moving rearward with the sheath. After the stent is released from the retention sheath, the stent springs radially outward to an expanded diameter until the stent contacts and presses against the vessel wall. Traditionally, self-expanding stents have been used in a number of peripheral arteries in the vascular system due to the elastic characteristic of these stents. One advantage of self-expanding stents for peripheral arteries is that traumas from external sources do not permanently deform the stent. As a result, the stent may temporarily deform during unusually harsh traumas and spring back to its expanded state once the trauma is relieved. However, self-expanding stents may be used in many other applications as well.

In the case where the distal tip is bonded to the inner catheter, a bead of adhesive may be applied to the interface between the proximal end of the distal tip and the inner catheter, thereby providing a smooth transition surface between the inner catheter and the distal tip. This smooth transition surface helps to minimize the risk of catching the stent or otherwise interfering with the stent's deployed position when the distal tip is withdrawn. In order to accommodate the bonding process and to provide the necessary space to apply the bead of adhesive, an undesirable gap may be introduced between a distal end of a stop attached to the inner catheter and the proximal end of the stent. When the retention sheath is withdrawn, the stent initially moves proximally with the retention sheath through the gap until the proximal end of the stent contacts the distal end of the stop. Once the proximal end of the stent contacts the distal end of the stop, the stop prevents the stent from continuing to move proximally, thereby resulting in relative movement between the stent and the retention sheath. However, because the stent initially moves proximally with the retention sheath through the gap, a slight delay in deployment may occur. This delay in deployment may cause inaccuracy in placement of the stent.

After the stent has been deployed, the inner catheter, including the distal tip, is withdrawn. As described above, the largest portion of the distal tip is typically larger than the outside diameter of the stent in its compressed form. Thus, provided that the inner diameter of the radially expanded stent is sufficiently greater than the maximum outer diameter of the distal tip, the distal tip of the inner catheter can be withdrawn through the stent without significant risk of dislodging or otherwise interfering with the placement or orientation of the deployed stent.

The distal tip generally performs the function of providing an atraumatic surface for the delivery catheter, which may assist in insertion through, or dilation of a stenosis. Without such a surface, the delivery catheter, or the stent may engage and damage the vessel wall or prevent insertion of the delivery sheath. However, in some circumstances it may not be preferred or possible to utilize a distal tip that is larger in diameter than the outer diameter of the stent in its compressed form, and smaller in diameter than the inner diameter of the stent in its expanded form, such that the inner catheter and the distal tip can be withdrawn safely and reliably through the center of the stent after deployment. For example, a distal tip may be impractical for delivery systems designed for use in vessels that are too small to accommodate a distal tip that is larger in diameter than the outer diameter of the stent in its compressed form. Additionally, distal tips that are larger in diameter than the outer diameter of the stent in its compressed form, and smaller in diameter than the inner diameter of the stent in its expanded form may also be impractical in delivery systems for stents having a small size differential between their expanded and compressed forms because the risk of the distal tip interfering with the placement of the stent upon retraction is high.

Moreover, in cases where the stent is deployed over a curved section(s) of a vessel, the risk of a distal tip disturbing the placement of a deployed stent upon withdrawal is exacerbated because the inner catheter is likely to contact the stent as it is retracted through the curved vessel. Furthermore, as delivery system profiles become increasingly smaller, the stent wall thickness, which contributes to the radially outward force the stent is capable of exerting against the vessel wall, may have to be reduced in order to accommodate the inner catheter, thereby potentially compromising the radially outward force exerted by the stent. Therefore, it has become apparent to the inventor that an improved delivery system that can be withdrawn safely and reliably without interfering with the placement of the stent is desirable.

The above-described examples are only some of the applications in which stents are used by physicians. Many other applications for stents or other implantable medical devices are known and/or may be developed in the future.

### SUMMARY

Delivery systems are described below that may allow for safe, more reliable placement of implantable medical devices. The invention may include any of the following aspects in various combinations and may also include any other aspect described below in the written description or in the attached drawings. In one embodiment, a delivery system includes a retention sheath for an implantable medical device. The retention sheath includes a central lumen extending from a proximal end to a distal end of the retention sheath, and a tapered portion disposed at a distal end of said retention sheath. The tapered portion may include a first layer made of a low-friction material, and the first layer may be movable from a compressed folded configuration in an initial position, to a substantially uncompressed and unfolded configuration in a deployment position.

The tapered portion may also include a second layer made of an expandable material. The second layer may be disposed radially outward of and in contact with the first layer. The second layer may also be configured to expand in a substantially radially outward direction when the first layer moves from the initial position to the deployment position. Additional details and advantages are described below in the detailed description.

In another embodiment, the delivery system may include an implantable medical device disposed within the central lumen of the retention sheath, thereby restraining the implantable medical device. In one aspect, the delivery system may include an inner catheter disposed within the central lumen of the retention sheath. The inner catheter may not extend into the tapered portion of the retention sheath.

A method of manufacturing a delivery system for an implantable medical device may include providing a retention sheath having a first layer including a lubricious material. A second layer including an expandable material is applied to an outer surface of the first layer, and a tapered portion is formed at the distal end of the retention sheath. The tapered portion may be formed by heating and compressing the second layer, and causing the first layer to form at least one fold underneath the second layer in the tapered portion.

In another aspect, the first layer may form a plurality of folds in a bunched configuration when the distal end of the retention sheath is molded to form the tapered portion.

The foregoing paragraphs have been provided by way of general introduction, and are not intended to limit the scope of the following claims. The presently preferred embodiments, together with further advantages, will be best understood by reference to the following detailed description taken in conjunction with the accompanying drawings.

### BRIEF DESCRIPTION OF THE DRAWINGS

The invention may be more fully understood by reading the following description in conjunction with the drawings, in which:

FIG. 1(a) is a side view of an implantable medical device delivery system in an undeployed state;

FIG. 1(b) is a side cross-sectional view of a distal portion of the implantable medical device delivery system of FIG. 1(a) in an initial position;

FIG. 1(c) is a side view of the implantable medical delivery system of FIG. 1(a) in a deployed position;

FIG. 2 is a perspective view of the delivery system of FIG. 1(b) in an initial position;

FIG. 3 is a cross-sectional view along the line X-X of the delivery system of FIG. 1(b) in an initial position;

FIG. 3(a) is a cross-sectional view along the line X-X of an alternative configuration of the delivery system of FIG. 1(b) in an initial position;

FIG. 4 is a perspective view of the delivery system of FIG. 1(b) in a partially deployed position;

FIG. 5 is a cross-sectional view along the line X-X of the delivery system of FIG. 4 in a partially deployed position;

FIG. 6 is a partial cross-sectional view of the delivery system of FIG. 1(b) in an undeployed state and positioned in a body passageway;

FIG. 7 is a partial cross-sectional view of the delivery system of FIG. 6 in a partially deployed state;

FIG. 8 is a partial cross-sectional view of the delivery system of FIG. 7 in a completely deployed state; and

FIG. 9 is a side cross-sectional view of an alternative embodiment of the distal portion of the implantable medical device delivery system of FIG. 1(b) in an initial position.

### DETAILED DESCRIPTION

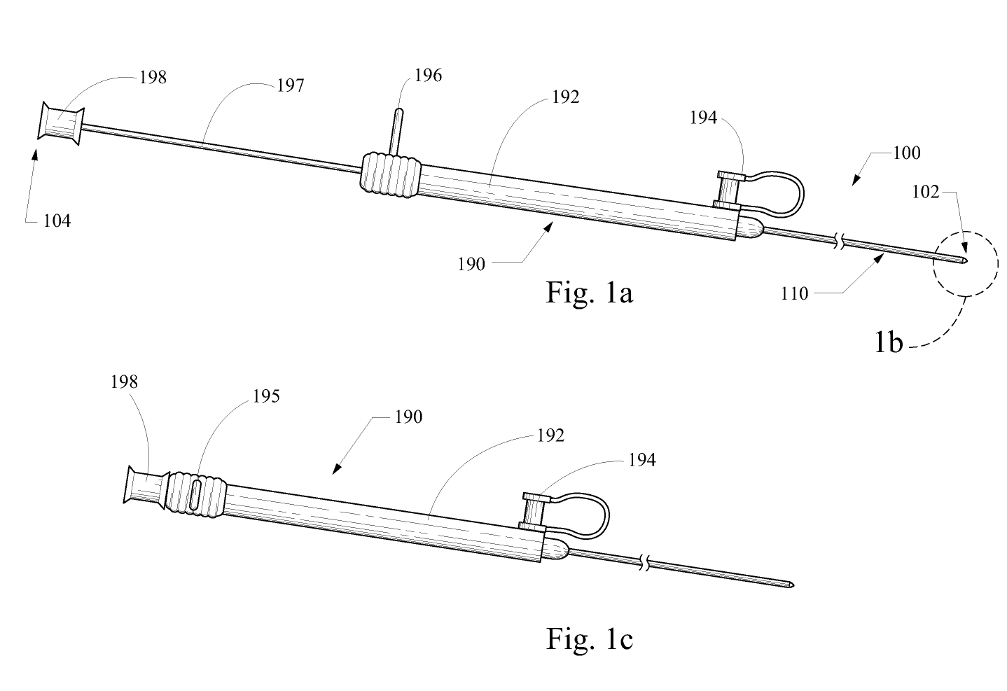

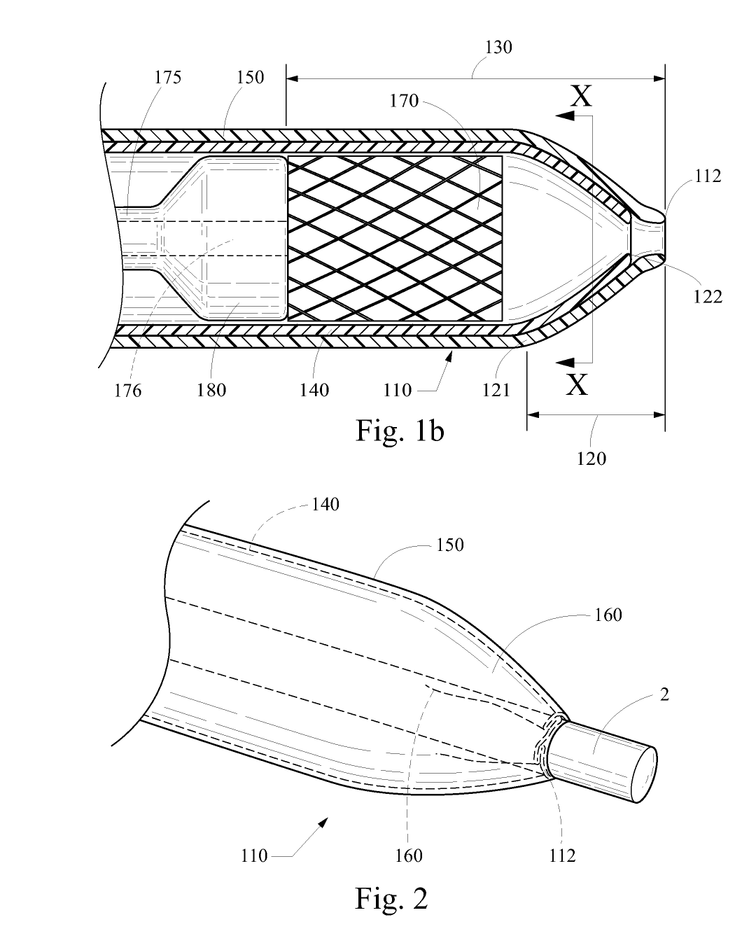

The term “axial” refers to the lengthwise direction 1 between the distal end 102 and the proximal end 104 of an implantable medical device delivery system 100. The axial direction is aligned with a central axis of the delivery system as shown in the Figures. The terms “distal” and “forward,” and variations thereof refer to the position or orientation relative to the distal end 102, of an implantable medical device delivery system, which is configured to receive a guide-wire and be inserted into a patient's vasculature, while the term “proximal” and “rearward,” and variations thereof refer to the position or orientation relative to the proximal end 104 of the delivery system 100, as shown in FIG. 1(a). The term implantable medical device refers to medical devices capable of being implanted within a human being including for example and without limitation, self-expanding stents, balloon expanding stents, coils, filters, baskets, valves, and endovascular grafts used in the treatment of patients, such as, for example, the treatment of arterial stenoses, aneurysms, and other minimally invasive procedures. While the following description of the embodiments of the present invention will be made with regard to self-expanding stents, it should be understood that the present invention is not limited thereto.

Referring now to the figures, FIG. 1(a)-2 illustrate an improved delivery system 100 for an implantable medical device. The implantable medical device may be, for example, and without limitation, a self-expanding stent. Various designs known in the art may be used for the self-expanding stent 170. For example, the self-expanding stent 170 may be made with serpentine rings interconnected with longitudinal struts. The stent 170 may also be made from a braided framework of wire filaments. Other well-known stent structures are also possible. Various materials may be used for the self-expanding stent 170, such as nitinol and stainless steel.

The delivery system 100 includes a retention sheath 110, a self-expanding stent 170, and a control device 190. The retention sheath 110 includes a distal portion 130 and a distal end 112. The distal portion 130 includes an inner layer 140, an outer layer 150, and a tapered portion 120. The control device 190 may include a control knob 198, a hollow shaft 197, a locking tab 196, a slot 195, a control handle 192, and a port 194. However, it should be understood that the control device 190 of the delivery system 100 is not limited thereto, and any control device configured to retract a retention sheath, as is known in the art, may be used.

In one embodiment, the delivery system may also include an inner catheter 175 disposed within an inner lumen of the retention sheath 110. The inner catheter 175 includes a stop 180 having proximal and distal surfaces, and a guide-wire lumen 176. The stop 180 is preferably disposed at the distal end of the inner catheter 175, and may be an integral part of the inner catheter 175 or a separate component that is bonded to, or otherwise affixed to the inner catheter 175, as is known in the art. It should be noted that the inner catheter 175 preferably does not include a distal tip.

The guide-wire lumen 176 extends through the center of the inner catheter 175 in an axial direction from the stop 180, to the proximal end of the inner catheter 175. A proximal portion of the inner catheter is disposed within a lumen extending through the center of the control handle 190, the shaft 197, and the control knob 198. A proximal end of the inner catheter 175 is fixedly attached to the control knob 198.

Preferably, the distal end of the inner catheter 175 terminates at the stop 180, which is disposed rearward of the proximal end of the stent 170, and the inner catheter 175 does not extend through the space defined by the inner diameter of the compressed stent 170. Because the inner catheter 175 does not protrude into the stent 170, the wall thickness of the stent 170 is only limited by the space between the inner surface of the retention sheath and the outer surface of the guide-wire 2 that the delivery system 100 is configured to receive. Thus, as compared to conventional implantable medical device delivery systems having the same outer diameter or “package size,” the delivery system 100 is able to accommodate thicker-walled stents capable of producing greater radially outward force against a vessel wall, larger guide-wires 2, or a combination thereof. Alternatively, the outer diameter of the delivery system 100 may be reduced.

The control handle 192 is disposed around the shaft 197 and is slideably movable relative to the shaft 197 in a proximal-distal direction from an initial position, in which the distal end of the control knob 198 is spaced axially away from the proximal end of the control handle 192 in an extended configuration, as shown in FIG. 1(a), to a deployment position in which the distal end of the control knob 198 is disposed adjacent the proximal end of the control handle 192, as shown in FIG. 1(c). The proximal end of the retention sheath 110 is connected to the control handle 192 at the distal end of the control handle 192.

The locking tab 196 may be inserted into the slot 195 and is configured to engage the shaft 197 such that when the locking tab 196 is inserted into the slot 195, the shaft 197 cannot move relative to the control handle 192, thereby preventing inadvertent or premature deployment of the stent 170.

The port 194 may be provided on the control handle to pass fluids, e.g. contrast fluid, through the delivery system to the treatment site. Preferably, the port 194 is in communication with the annular space between the inner catheter 175 and the retention sheath 110, however, it should be understood that the port 194 may be in communication with the guide-wire lumen 176 of the inner catheter 175 or a lumen disposed within the retention sheath 110.

The stent 170 is disposed at the distal end 122 of the retention sheath 110 in a compressed configuration, such that the stent 170 exerts a radially outward force against the inner surface of the retention sheath 110, and the retention sheath 110 restrains the stent 170 in the compressed configuration.

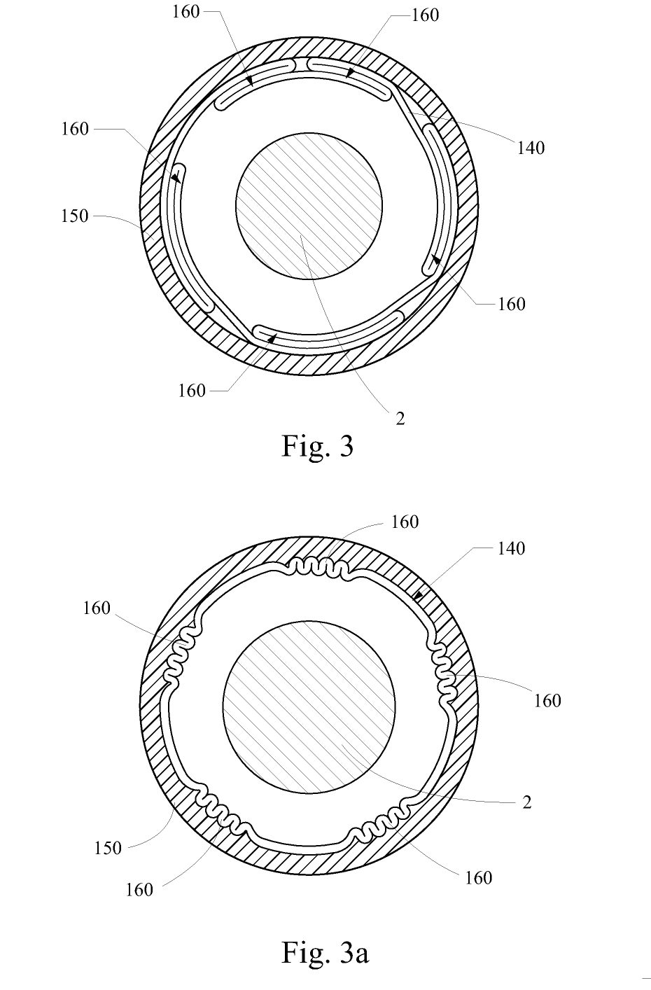

The retention sheath 110 has an outer diameter and an inner surface that defines the inner lumen extending axially from a proximal end, which is attached to the control handle 192, to the distal end 112 of the retention sheath 110. Because the tapered portion 120 of the retention sheath 110 is configured to expand and slide over the stent 170 during retraction and deployment, it is desirable that the tapered portion 120 possess both high elasticity, or stretchability/expandability and a low coefficient of friction. Unfortunately, these two properties rarely coincide in the same material. For example, materials such as PTFE that are customarily employed to provide high lubricity or low friction, do not exhibit good expandability. Similarly, low durometer materials such as Nylon, polyester block amide or PEBAX (polyether block amide), which possess the required expandability, do not offer high lubricity or low friction. Thus, the retention sheath 110, and in particular the distal portion 130, may be a composite of different materials, the base material of which is preferably made from a lubricious material, for example PTFE (polytetrafluoroethylene) or the like. The retention sheath 110 also may incorporate wire coils or braids to increase the sheath's resistance to torsional and compressive forces. However, in embodiments incorporating wire coils or braids, it is preferable that the wire coils or braids do not extend into the tapered portion 120 of the distal portion 130 to facilitate the creation of folds 160 in the tapered section 120 of the inner layer 140, as shown in FIGS. 2 and 3.

The distal portion 130 of the retention sheath 110 extends proximally from the distal end 112 of the retention sheath 110 in a dual layer construction comprised of an inner layer 140 and an outer layer 150. The distal portion 130 may terminate at the proximal end of the tapered section 120, or at any intermediate point between the proximal end of the tapered section 120 and the proximal end of the retention sheath 110. Alternatively, the entire sheath may incorporate the dual layer construction; that is, the inner layer 140 and the outer layer 150 may extend from the proximal end of the retention sheath 110 that is connected to the control handle 192, to the distal end 112 of the retention sheath 150. The inner layer 140 is disposed at the radially inward most portion of the retention sheath 110 such that an inner surface of the inner layer 140 forms the inner lumen of the retention sheath 110. The inner layer 140 is made of a low-friction or lubricious material that is generally inelastic, and is preferably an extension of the PTFE base material of the retention sheath 110. It should be understood that other low-friction or lubricious materials may be used for the inner layer 140, as is known in the art.

The outer layer 150 is disposed around the inner layer 140 such that an inner surface of the outer layer 150 contacts the outer surface of the inner layer 140. The outer layer 150 is preferably made of a low-durometer expandable material that forms the tapered portion 120, for example and without limitation, Nylon, polyether block amide, and polyester block amide. The tapered portion 120 preferably extends in a smooth transition from a large outer diameter 121 disposed adjacent the distal end of the stent 170, to a small outer diameter 122 disposed at the distal end 112 of the retention sheath 110. However, it should be understood that the tapered portion 120 is not limited thereto, and may transition from the large outer diameter 121 to the small outer diameter 122 in an undulating and non-smooth manner provided that the transition results in the tapered portion 120 having an atraumatic profile. Furthermore, it should be understood that the large outer diameter 121 may be disposed forward of the distal end of the stent 170.

Preferably, the tapered portion 120 extends about two millimeters forward of the distal end of the stent 170. However, the tapered portion 120 may extend less than two millimeters forward of the distal end of the stent 170, or may extend up to 10 millimeters forward of the distal end of the stent 170. As shown in FIG. 9, in an alternative embodiment, the distal end 151 of the outer layer 150 may extend slightly past the distal end 141 of the inner layer 140 in the distal direction, such that the portion of the outer layer 150 extending past the distal end of the inner layer 140 contacts the outer surface of the stent 170 as the retention sheath 110 is retracted during deployment.

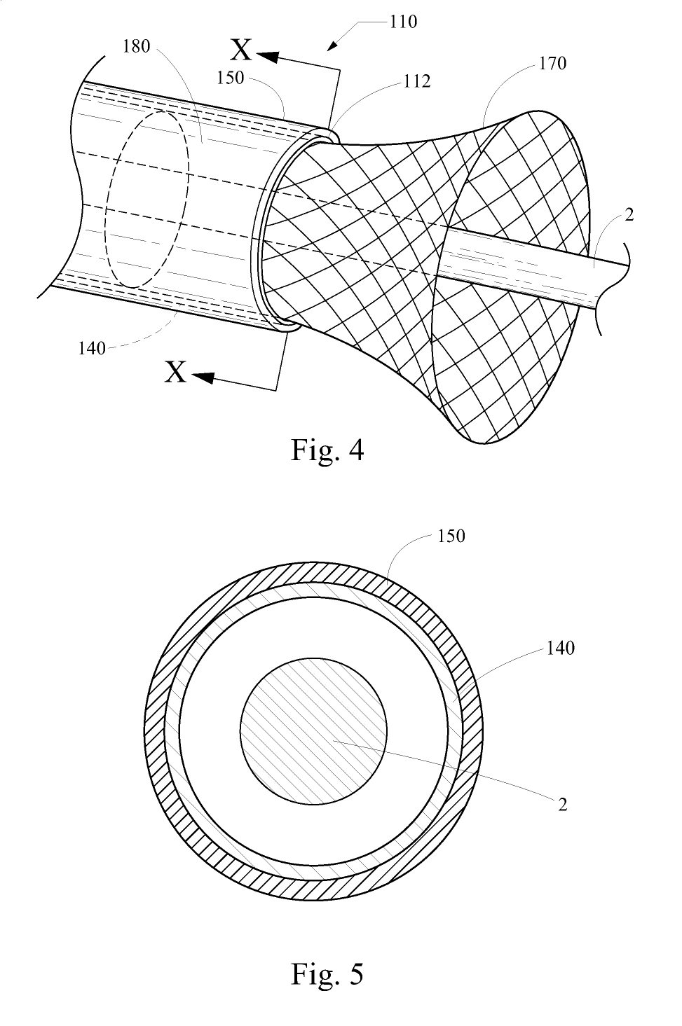

The tapered portion 120 of the distal portion 130 is preferably formed by applying the outer layer 150 over the inner layer 140 and drawing the outer layer 150 down to form a tapered shape. Therefore, prior to drawing the outer layer 150 down and forming the tapered shape, the inner layer 140 has a substantially constant inner diameter throughout the distal portion 130. Similarly, the outer layer 150 preferably has a substantially constant inner diameter that is substantially equivalent to the outer diameter of the inner layer 140 throughout the distal portion 130, prior to drawing the outer layer 150 down to form the tapered shape. Thus, prior to forming the tapered shape, the outer layer 150 and the inner layer 140, may have a configuration similar to the partial deployment configuration shown in FIGS. 4 and 5. Note that the outer diameter of the outer layer 150 may increase in the distal direction through the tapered portion 120 to ensure that the wall thickness of the outer layer 150 is sufficient to hold the inner layer 140 in a compressed, folded configuration and to provide an atraumatic surface of sufficient strength to dilate a stenosis after the outer layer 150 is formed into the tapered shape. For example, the wall thickness of the outer layer 150 after being formed into the tapered shape may be greater than or equal to 0.0001 inches.

The material properties of the outer layer 150 allow the outer layer 150 to compress and flow around the inner layer 140 as the outer layer 150 is drawn down to form the tapered shape. However, because the inner layer 140 is generally inelastic and not readily expandable, as the outer layer 150 transitions from the large outer diameter 121 to the small outer diameter 122, the inner layer 140 is forced to assume a folded configuration in the tapered portion 120 in order to accommodate the tapered profile of the outer layer 150. As shown in the cross-sectional view of FIGS. 3 and 3(a), this folded configuration may include a plurality of folds 160 that result in a bunching or puckering of the inner layer 140 in the tapered portion 120. Alternatively, the folded configuration of the inner layer 140 may include a single fold in the tapered portion 120. However, it should be understood that any that any number, shape, or configuration of the folds 160 is acceptable, provided that the inner layer 140 is able to conform to the tapered shape of the outer layer 150 in the tapered portion 120.

As shown in FIG. 2, as the outer layer 150 transitions from a large outer diameter 121 to a small outer diameter 122 in the distal direction, the degree to which the inner layer 140 folds in on itself gradually increases from a minimum, disposed at the proximal end of the tapered portion 120, to a maximum, disposed at the distal end of the tapered portion 120, for each fold 160.

In operation, initially, the guide-wire 2 is advanced through a trocar into a desired vessel or cavity using the Seldinger technique which is conventional and well known in the art. The guide-wire is then advanced through the patient's vasculature or cavity until it reaches the desired treatment site. Once the guide-wire 2 is in the desired position, a proximal end of the guide-wire 2 is inserted into the distal end of the guide-wire lumen 176. The delivery system 100 is then inserted into a patient's vasculature or cavity by sliding the delivery system 100 along the guide-wire 2 in a distal direction.

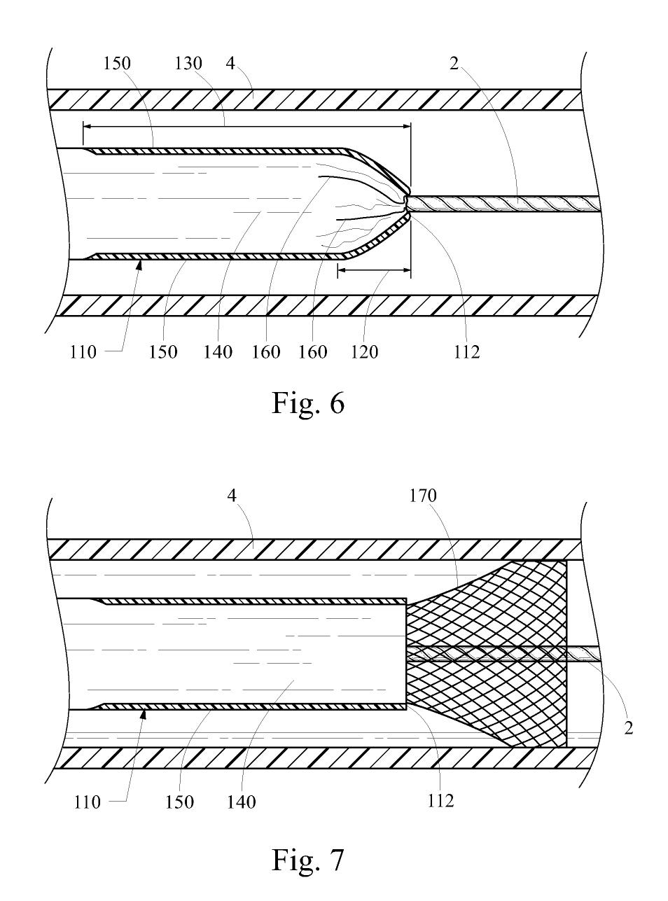

Referring to FIG. 6, as the delivery system 100 is moved in the distal direction, it is guided through the patient's vasculature by the guide-wire 2 to a treatment site, for example, a stenosis. The stent 170 may be positioned at the treatment site using radiopaque markers located on the stent 170. The radiopaque markers allow a physician to visualize the stent 170 from outside the patient's body using x-ray fluoroscopy.

Once the stent 170 is in position at the treatment site, the physician pulls the control handle 192 toward the control knob 198, which causes the retention sheath 110 to move in the proximal direction relative to the inner catheter 175. Due to frictional forces caused by the outward radial force of the compressed stent 170 against the inner surface of the retention sheath 110, a portion of the retraction force applied at the control handle 192 is transferred to the stent 170, thereby forcing the proximal end of the stent 170 against the distal surface of the stop 180.

As illustrated in FIGS. 4-8, as the physician continues to pull the control handle 192 in the proximal direction, the retention sheath 110 is retracted in the proximal direction, and the stop 180 provides a reaction surface for the stent 170, thereby substantially preventing the stent 170 from moving in the axial direction toward the control device 190. As the retention sheath is moved proximally relative to the stent 170, the distal end of the stent 170 contacts the inner surface of the inner layer 140 at the tapered portion 120, and forces the folds 160 of the inner layer 140 to unfold, thereby causing the outer layer 150 to expand in a radially outward direction as the inner layer 140 assumes its unfolded and uncompressed configuration, as shown in FIGS. 4 and 5. Thus, as the retention sheath 110 is retracted, the stent 170 contacts only the low-friction, lubricious material of the inner layer 140, thereby minimizing friction and facilitating retraction of the retention sheath 110. It should be understood that the outer layer 150 may be made of an elastic material that expands outward during deployment and that may substantially return to the initial tapered configuration after deployment of the stent 170.

In addition to minimizing friction between the stent 170 and the retention sheath 110 during deployment, the dual layer construction of the distal portion 130 also aids in retention and compression of the stent 170 as the generally inelastic and not readily expandable inner layer 140 maintains a substantially constant inner diameter in the portions contacting the stent 170 before, during, and after deployment. Because the inner layer 140 extends to the distal end 112 of the retention sheath 110 and the inner diameter of the inner layer 140 retains the substantially constant inner diameter during deployment, the retention sheath is retracted evenly around the circumference of the stent 170 as the control handle 192 is moved in the proximal direction. Thus, the stent 170 is released in a controlled and uniform manner around the circumference of the stent 170, which aids in proper and precise placement of the stent 170.

In embodiments in which the outer layer 150 extends past the distal end of the inner layer 140, the extended portion of the outer layer 150 contacts and grips the stent 170 as the retention sheath 110 is withdrawn. As the stent 170 expands, the stent 170 forces the distal most portion of the outer layer 150 to expand in a radially outward direction, thereby minimizing the effects of friction on deployment. However, because a small portion of the low durometer outer layer 150 is in contact with the stent 170, the stent 170 is less likely to “jump” slightly in the distal direction, thus allowing for more accurate and reliable placement.

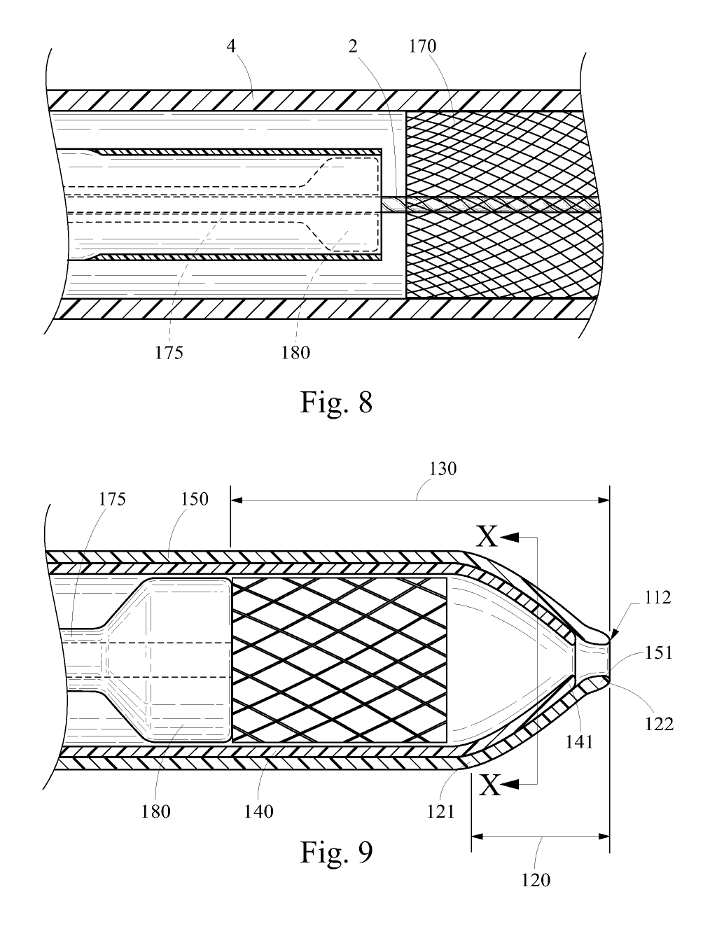

As shown in FIG. 8, once the stent 170 is completely deployed, the entirety of the delivery system 100 is located rearward of the stent 170, and the delivery system can be withdrawn without the risk of disturbing the placement of the stent 170. Thus, the delivery system 100 provides an atraumatic surface disposed at the distal end of the delivery system that prevents inadvertent damage to the vessel wall and assists in dilation of a stenosis or the like during insertion, yet minimizes the risk of disturbing the placement of the stent 170 during withdrawal.

The delivery system 100 possesses significant advantages over conventional delivery systems utilizing inner catheters that extend through the center of the stent 170, and particularly over delivery systems that utilize inner catheters having distal tips, which may come in contact with the stent 170 and interfere with the stent's placement during removal. Additionally, the lack of a conventional distal tip forward of the stenosis, may be advantageous in small vessels. In some cases, the lack of a conventional distal tip may also be advantageous in manufacturing in that there is no distal tip to be added as a final step in production of the inner catheter 175.

The improved retention sheath may be manufactured by initially forming an inner layer 140 made of a lubricious or low-friction material, such as PTFE, such that the distal end of the inner layer 140 will extend past the distal end of the stent 170 after insertion. Preferably, the inner layer 140 is formed as an integral portion of the base layer of the retention sheath 110 that extends past the distal end of the stent 170 after insertion into the retention sheath 110. However, it should be understood that the stent 170 is preferably not inserted into the retention sheath 110 at this point. Once the inner layer 140 has been formed, the outer layer 150, which is preferably made of a heat formable material, for example, a thermoplastic polymer, such as Nylon, is preferably applied only to the distal portion 130 of the retention sheath 110. Alternatively, the thermoplastic polymer may be applied to any intermediate portion between the proximal end of the tapered section 120 (prior to forming the taper) and the proximal end of the retention sheath 110, or the outer surface of the entire sheath. After the outer layer 150 has been applied, the outer layer 150 and the inner layer 140, along with a wire coil or braid may be fused together as described in U.S. Pat. Nos. 5,380,304, and 5,700,253, which are assigned to Cook Incorporated, the assignee of the present invention, and are hereby incorporated by reference in their entirety.

After the inner and outer layers 140, 150 have been fused together, the stent 170 is inserted into the retention sheath 110 from either the proximal or distal end. The tapered portion 120 is then formed, preferably by heating the tapered portion 120 of the retention sheath 110 to the workable range of Nylon, which is between 356 to 500 degrees Fahrenheit, and significantly below the melting point of the PTFE inner layer 140 of 620.6 degrees Fahrenheit. Preferably, the tapered portion 120 is heated to 365 degrees Fahrenheit and then compressed in a mold to achieve a tapered shape having a smooth transition from the large outer diameter 121 to the small outer diameter 122. However, it should be understood that the tapered portion 120 may be formed using other methods, as is known in the art. As described above, the thickness of the outer layer 150 may increase in the distal direction to compensate for the flow of the outer layer 150 material during the forming process of the tapered section 120. Because the tapered portion is heated to a temperature below the melting point of the inner layer 140, the inner layer 140 does not melt and is forced into a folded configuration by the mold as the Nylon outer layer 140 flows around the outer surface of the inner layer 140 and conforms to the shape of the mold. The folded configuration of the inner layer 140 may include a plurality of folds 160 that result in a bunching or puckering of the inner layer 140 in the tapered portion 120, as shown in the cross-sectional views of FIGS. 3 and 3(a). Alternatively, the folded configuration of the inner layer 140 may include a single fold in the tapered portion 120. However, it should be understood that provided that the inner layer 140 is able to conform to the tapered shape of the outer layer 150 in the tapered portion 120, any number, shape, or configuration of the folds 160 is acceptable.

Although the majority of the preceding detailed description has been made with reference to self-expanding stents, it should be understood that the delivery system of the present invention is not limited thereto, and may be used for any number of implantable medical devices, including for example and without limitation, occluding devices, balloon expanding stents, coils, valves, or filters.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A delivery system for an implantable medical device, comprising:
a retention sheath comprising a central lumen, and a tapered portion disposed at a distal end of said retention sheath, said tapered portion comprising:
(a) a first layer comprising a low-friction material, wherein said first layer is movable from an initial position where said first layer is in a compressed folded configuration, to a deployment position where said first layer is in a substantially uncompressed and unfolded configuration; and
(b) a second layer comprising a stretchable material, wherein said second layer is disposed radially outward of said first layer, and wherein said second layer is configured to expand in a substantially radially outward direction when said first layer moves from said initial position to said deployment position;
an implantable medical device disposed within said central lumen of said retention sheath, said retention sheath restraining said implantable medical device; and
an inner catheter disposed within said central lumen of said retention sheath, wherein said inner catheter does not extend into said tapered portion of said retention sheath.

2. The delivery system of claim 1, wherein said implantable medical device is a self-expanding stent.

3. The delivery system of claim 1, wherein said inner catheter terminates rearward of a proximal end of said implantable medical device.

4. The delivery system of claim 1, wherein said first layer is a lubricious material.

5. The delivery system of claim 4, wherein said lubricious material is polytetrafluoroethylene.

6. The delivery system of claim 1, wherein said second layer is a heat formable material.

7. The delivery system of claim 6, wherein said heat-formable material is a thermoplastic polymer.

8. The delivery system of claim 7, wherein said thermoplastic polymer material is selected from one of the group of Nylon, polyether block amide, and polyester block amide.

9. The delivery system of claim 1, wherein said second layer extends distally beyond a distal end of said first layer.

10. The delivery system of claim 1, wherein said inner catheter terminates rearward of a proximal end of said implantable medical device, said first layer is a lubricious material, and said second layer is a heat-formable material.

11. The delivery system of claim 10, wherein said lubricious material is polytetrafluoroethylene, said heat-formable material is selected from one of the group of Nylon, polyether block amide, and polyester block amide, and said second layer extends distally beyond a distal end of said first layer.

12. A retention sheath for an implantable medical device, said retention sheath comprising:
a central lumen extending from a proximal end to a distal end of said retention sheath, and a tapered portion disposed at a distal end of said retention sheath, said tapered portion comprising:
(a) a first layer comprising a low-friction material, wherein said first layer is movable from a compressed folded configuration in an initial position, to a substantially uncompressed and unfolded configuration in a deployment position; and
(b) a second layer comprising an expandable material, wherein said second layer is disposed radially outward of and in contact with said first layer, and wherein said second layer is configured to expand in a substantially radially outward direction when said first layer moves from said initial position to said deployment position.

13. The retention sheath of claim 12, wherein said first layer is a lubricious material.

14. The retention sheath of claim 13, wherein said lubricious material is polytetrafluoroethylene.

15. The retention sheath of claim 12, wherein said expandable material is a thermoplastic polymer selected from one of the group of Nylon, polyether block amide, and polyester block amide.

16. A method of manufacturing an implantable medical device delivery system, said method comprising:
providing a retention sheath having a first layer, said first layer comprising a lubricious material;
applying a second layer to an outer surface of said first layer, said second layer comprising an expandable material;
forming a tapered portion disposed at said distal end of said retention sheath, wherein said tapered portion is formed by heating and compressing said second layer and causing said first layer to form at least one fold underneath said second layer in said tapered portion of said retention sheath.

17. The method of claim 16, wherein said first layer forms a plurality of folds in a bunched configuration when said distal end of said retention sheath is molded to form said tapered portion.

18. The method of claim 16, further comprising inserting an implantable medical device into said distal end of said retention sheath prior to molding said tapered portion.

19. The method of claim 16, wherein said implantable medical device is a self-expanding stent.

20. The method of claim 16, further comprising:
Inserting an implantable medical device into said distal end of said retention sheath prior to molding said tapered portion,
wherein said implantable medical device is a self-expanding stent, said lubricious material is polytetrafluoroethylene, and said expandable material is selected from one of the group of Nylon, polyether block amide, and polyester block amide.

21. The method of claim 16, further comprising extending said second layer distally beyond a distal end of said first layer.
